(19) United States
(12) Patent Application Publication
(10) Pub. No.: US 2026/0340819 A1
(43) Pub. Date: Oct. 29, 2026

(54) SYSTEM FOR MEDICAL IMAGING QUALITY CONTROL AND ORGAN PHENOTYPE EXTRACTION

(71) Applicants: The First Affiliated Hospital of Gannan Medical University, Ganzhou (CN); Shenzhen Jimmy Research Co., LTD, Shenzhen (CN)

(72) Inventors: Yi XIANG, Ganzhou (CN); Gangfeng ZHU, Ganzhou (CN); Yi ZHOU, Ganzhou (CN); Beijia YU, Ganzhou (CN); Yi XIE, Ganzhou (CN); Yipeng SONG, Ganzhou (CN); Li HUANG, Ganzhou (CN); Zenghong LU, Ganzhou (CN); Rui ZHU, Ganzhou (CN); Junyan LI, Ganzhou (CN)

(73) Assignees: The First Affiliated Hospital of Gannan Medical University, Ganzhou (CN); Shenzhen Jimmy Research Co., LTD, Shenzhen (CN)

(21) Appl. No.: 19/277,459

(22) Filed: Jul. 23, 2025

(30) Foreign Application Priority Data
Apr. 28, 2025 (CN) 202510548612.2

(51) Int. Cl. G06T 7/00; G06T 7/12; G06T 7/155; G06T 7/162; G06V 10/26; G16H 30/20; G16H 30/40

(52) U.S. Cl. CPC G06T 7/0012; G06T 7/12; G06T 7/155; G06T 7/162; G06V 10/26; G16H 30/20; G16H 30/40; G06T 2207/20036; G06T 2207/30004; G06T 2207/30168; G06V 2201/031

(57) ABSTRACT

A system for medical imaging quality control and organ phenotype extraction is provided. A topological relationship tree of organ phenotypes is constructed according to association of anatomical structures between various organ phenotypic characteristics in a medical image, and a structural association graph of organ phenotypes in the medical image is determined; a semantic structure graph of medical entities in a text report is determined through a logical association relationship between the medical entities and a contextual relationship of clinical description statements in the text report; node-level alignment and fusion are performed on the structural association graph and the semantic structure graph of medical entities to obtain an organ-entity characteristic association map; medical imaging data is quality-controlled based on the characteristic association map, and a quality control warning is output if it is determined that the medical imaging data has an imaging quality anomaly.

### CROSS-REFERENCE TO THE RELATED APPLICATIONS

This application is based upon and claims priority to Chinese Patent Application No. 202510548612.2, filed on Apr. 28, 2025, the entire contents of which are incorporated herein by reference.

### TECHNICAL FIELD

The present disclosure relates to the technical field of image analysis, and more specifically, the present disclosure relates to a system for medical imaging quality control and organ phenotype extraction.

### BACKGROUND

Medical imaging quality control is a key step to ensure imaging diagnosis accuracy. Conventional quality control relies on manual review, which has problems of highly subjectivity and inefficiency, and is prone to omissions of details and diagnostic bias. Image analysis technology automatically identifies image characteristics through algorithms, achieves objective quantitative evaluation of quality parameters such as imaging clarity, noise, and artifacts, improves detection consistency and efficiency, and can further automatically detect device failures or operating errors using deep learning, edge detection and other methods, thus reducing human intervention. Its application makes up for shortcomings of conventional quality control in standardization, real-time performance, and detail recognition capabilities, and significantly enhances accuracy and stability of medical imaging quality control.

Existing medical imaging quality control technologies mainly rely on independent evaluation of single-modal data, usually take image-level parameters such as clarity, noise, and artifacts as quality control basis, and ignore inherent association between images and multimodal data such as text reports and clinical descriptions, resulting in a lack of clinical context support for quality control results, and having a problem of accurate local evaluation but insufficient overall reliability. In addition, most existing methods do not fully utilize correspondence between medical entity information and imaging data in text reports, and fail to assist in verifying imaging quality from a semantic level. A quality control process has a problem of over-reliance on a single data source, which affects overall data credibility. Faced with defects such as insufficient fusion of multimodal data, limited understanding of organ phenotype structure, and ineffective use of clinical semantic information, existing technologies are difficult to meet needs of high-reliability medical imaging quality control. Therefore, how to achieve multimodal association quality control of medical imaging data so as to improve data reliability has becomes a difficult problem faced by the industry.

### SUMMARY

The present disclosure provides a system for medical imaging quality control and organ phenotype extraction, which can realize multimodal association quality control of medical imaging data, thereby improving reliability of the data.

In the first aspect, the present disclosure provides a system for medical imaging quality control and organ phenotype extraction, specifically including:
a data acquisition module, configured to collect medical imaging data to be tested for quality control, wherein the medical imaging data includes a medical image and a text report;
an organ phenotype extraction module, configured to construct a topological relationship tree of organ phenotypes according to association of anatomical structures between various organ phenotypic characteristics in the medical image, and then determine a structural association graph of the organ phenotypes in the medical image based on the topological relationship tree and distribution information of organ contours in the organ phenotypic characteristics;
a medical entity association module, configured to determine a semantic structure graph of medical entities in the text report through a logical association relationship between the medical entities and a contextual relationship of clinical description statements in the text report;
a characteristic fusion module, configured to perform node-level alignment and fusion on the structural association graph of the organ phenotypes and the semantic structure graph of the medical entities to obtain an organ-entity characteristic association map; and
a quality control determination module, configured to perform quality control determination on the medical imaging data based on the characteristic association map, and output a quality control warning if it is determined that the medical imaging data has an imaging quality anomaly.

In the present embodiment, the medical imaging data to be tested for quality control is collected from a hospital picture archiving and communication system (PACS) system.

In the present embodiment, a format of the medical image is digital imaging and communications in medicine (DICOM) format.

In the present embodiment, a format of the text report is health level seven (HL7) format.

In the present embodiment, constructing the topological relationship tree of the organ phenotypes according to the association of the anatomical structures between various organ phenotypic characteristics in the medical image specifically includes:
performing organ segmentation on the medical image using a pre-trained organ segmentation model, and extracting various organ phenotypic characteristics;
defining parent-child association relationships between various organ phenotypic characteristics based on a spatial position and anatomical proximity relationship of organs in a human body; and using the various organ phenotypic characteristics as nodes of the tree, and
using the parent-child association relationships between various organ phenotypic characteristics as edges of the tree, thereby obtaining the topological relationship tree of the organ phenotypes.

In the present embodiment, determining the structural association graph of the organ phenotypes in the medical image based on the topological relationship tree and the distribution information of the organ contours in the organ phenotypic characteristics specifically includes:
extracting the distribution information of the organ contours in various organ phenotypic characteristics;
determining attribute information of various organ phenotypes according to the distribution information of various organ contours;
determining attribute relationships between various organ phenotypic characteristics through all the attribute information; and
adjusting the edges in the topological relationship tree through all the attribute relationships, and using an adjusted topological relationship tree as the structural association graph of the organ phenotypes in the medical image.

In the present embodiment, determining the semantic structure graph of the medical entities in the text report through the logical association relationship between the medical entities and the contextual relationship of the clinical description statements in the text report specifically includes:
performing text entity recognition on the text report to extract the medical entities in the text report;
performing dependency parsing on the clinical description statements based on all the medical entities, to obtain the contextual relationship between the medical entities;
determining relationship coefficients between the medical entities through the contextual relationship between the medical entities and the logical association relationship between the medical entities; and
taking the medical entities as nodes and the relationship coefficients between the medical entities as edges, thereby obtaining the semantic structure graph of the medical entities in the text report.

In the present embodiment, performing the node-level alignment and fusion on the structural association graph of the organ phenotypes and the semantic structure graph of the medical entities to obtain the organ-entity characteristic association map specifically includes:
aligning corresponding nodes between the structural association graph of the organ phenotypes and the semantic structure graph of the medical entities to obtain multiple aligned and fused points;
performing semantic matching on the aligned structural association graph and semantic structure graph to obtain characteristic association values of various aligned and fused points; and
determining the organ-entity characteristic association map through all the characteristic association values.

In the present embodiment, performing the quality control determination on the medical imaging data based on the characteristic association map specifically includes:
performing modality association evaluation on an imaging quality of the medical imaging data through the characteristic association map to obtain a quality evaluation value of the medical imaging data; and
determining that the medical imaging data has an imaging quality anomaly if the quality evaluation value is lower than a preset imaging anomaly threshold.

In the present embodiment, outputting the quality control warning if it is determined that the medical imaging data has an imaging quality anomaly means that when the quality control determination module determines that the medical imaging data has the imaging quality anomaly, a warning notification is used to prompt relevant personnel that the imaging data has a quality problem.

The technical solutions provided by the embodiments disclosed in the present disclosure have the following beneficial effects:
firstly, collecting the medical imaging data to be tested for quality control, wherein the medical imaging data includes the medical image and the text report;FIG. 1100
constructing the topological relationship tree of the organ phenotypes according to association of anatomical structures between various organ phenotypic characteristics in the medical image, and then determining the structural association graph of organ phenotypes in the medical image based on the topological relationship tree and the distribution information of organ contours in the organ phenotypic characteristics;
determining the semantic structure graph of the medical entities in the text report through the logical association relationship between the medical entities and the contextual relationship of the clinical description statements in the text report;
performing the node-level alignment and fusion on the structural association graph of the organ phenotypes and the semantic structure graph of the medical entities to obtain the organ-entity characteristic association map; and
performing the quality control determination on the medical imaging data based on the characteristic association map, and outputting the quality control warning if it is determined that the medical imaging data has the imaging quality anomaly.

It can be seen that the solutions of the present disclosure perform the node-level alignment and fusion on the structural association graph of the organ phenotypes and the semantic structure graph of the medical entities to obtain the organ-entity characteristic association map, and then perform the quality control determination on the medical imaging data based on the characteristic association map. Firstly, the organ phenotype extraction module systematically models inter-organ phenotypic characteristics by constructing the organ phenotype topological relationship tree, and combining the anatomical structure association, effectively solves the problem of incomplete understanding of organ structure in the prior art, and further generates the structural association graph through the organ contour distribution information, so as to realize accurate identification of hidden imaging anomalies such as contour loss or dislocation, and improve meticulousness of image layer quality control. Secondly, the medical entity association module constructs the semantic structure graph based on the contextual relationship and the logical association of the medical entities in the text report to make up for a shortcoming of conventional methods that do not utilize text semantics for quality control verification, thus enabling imaging quality control results to have clinical context support, and enhancing clinical applicability and interpretability of quality control. The characteristic fusion module then fuses the structural association graph of the organ phenotypes with the semantic structure graph of the medical entities through the node-level alignment, establishes a cross-modal organ-entity characteristic association map, breaks through an information barrier between the imaging data and text data, solves a problem of insufficient multimodal data fusion, and realizes information complementarity. Finally, the quality control determination module performs quality control analysis based on the characteristic association map, thus being capable of integrating image quality and text semantic consistency, accurately identifying the imaging quality anomaly, avoiding deviation caused by single data source determination, and improving reliability and intelligence level of overall quality control determination.

In summary, the technical solutions of the present disclosure can realize multimodal association quality control of the medical imaging data, thereby improving the reliability of data.

### BRIEF DESCRIPTION OF THE DRAWINGS

In order to more clearly illustrate the technical solutions in the embodiments of the present disclosure or the prior art, the following will briefly introduce the drawings required for use in the embodiments or the prior art description. Obviously, the drawings described below are only for the embodiments of the present disclosure. For those ordinarily skilled in the art, other drawings could be obtained based on these drawings without using inventive efforts.

FIG. 1 is a flowchart of a system for medical imaging quality control and organ phenotype extraction provided by the present disclosure;

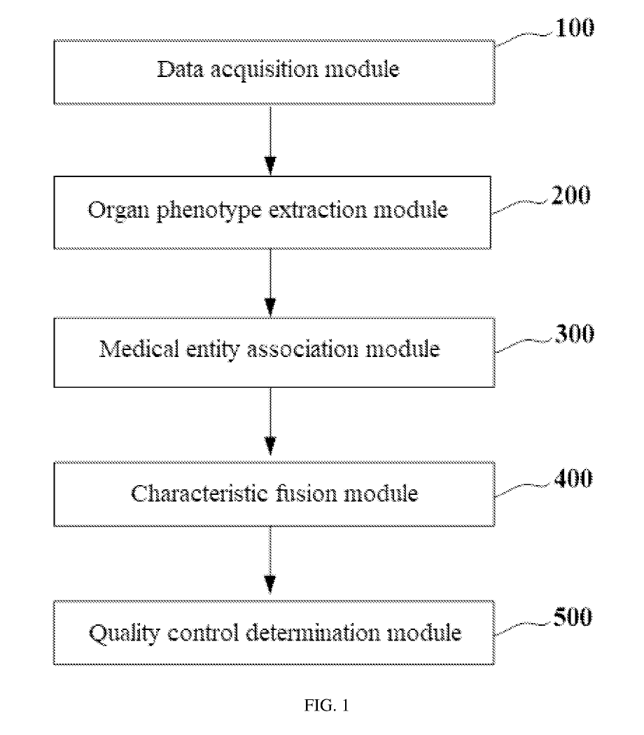

FIG. 2 is an exemplary flowchart for determining a topological relationship tree provided by the present disclosure; and

FIG. 3 is an exemplary flowchart for determining a semantic structure graph provided by the present disclosure.

### DETAILED DESCRIPTION OF EMBODIMENTS

The following will combine the drawings in embodiments of the present disclosure to clearly and completely describe technical solutions in the embodiments of the present disclosure. Obviously, the described embodiments are only part of the embodiments of the present disclosure, not all of the embodiments. Based on the embodiments in the present disclosure, all other embodiments obtained by those ordinarily skilled in the art without using inventive efforts are within the scope of protection of the present disclosure.

The embodiments of the present disclosure provide a system for medical imaging quality control and organ phenotype extraction, a core of which is to construct a topological relationship tree of organ phenotypes according to association of anatomical structures between various organ phenotypic characteristics in a medical image, and determine a structural association graph of organ phenotypes in the medical image based on the topological relationship tree and distribution information of organ contours in the organ phenotypic characteristics; determine a semantic structure graph of medical entities in a text report through a logical association relationship between the medical entities and a contextual relationship of clinical description statements in the text report; perform node-level alignment and fusion on the structural association graph of the organ phenotypes and the semantic structure graph of the medical entities to obtain an organ-entity characteristic association map; and perform quality control determination on medical imaging data based on the characteristic association map, and output a quality control warning if it is determined that the medical imaging data has an imaging quality anomaly. Based on the above solution, multimodal association quality control of the medical imaging data can be achieved, thereby improving reliability of the data.

It should be noted that the medical imaging data to be tested for quality control in the present disclosure can be collected from PACS of the hospital, and the medical imaging data includes the medical image and the text report, wherein a format of the medical image is DICOM format, and a format of the text report is HL7 format.

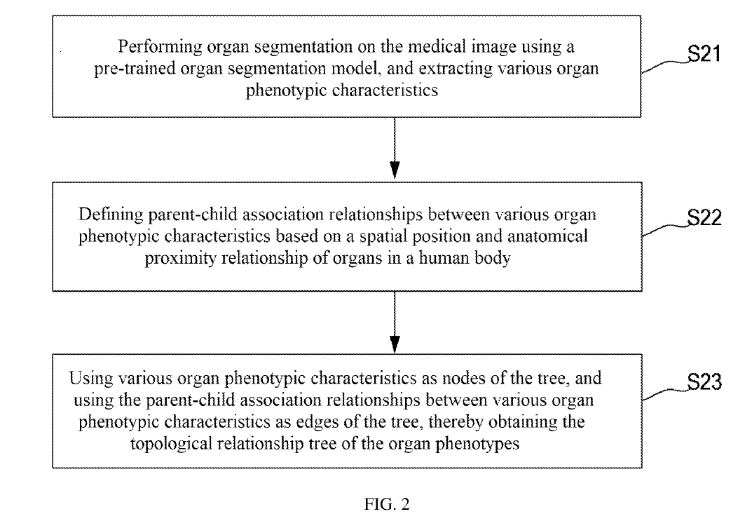

In the present embodiment, referring to FIG. 2, the figure is an exemplary flowchart for determining a topological relationship tree in an embodiment of the present disclosure. In the present embodiment, constructing the topological relationship tree of organ phenotypes according to the association of anatomical structures between various organ phenotypic characteristics in the medical image can be implemented by following steps:
in step S21, performing organ segmentation on the medical image using a pre-trained organ segmentation model, and extracting various organ phenotypic characteristics;
in step S22, defining parent-child association relationships between various organ phenotypic characteristics based on a spatial position and anatomical proximity relationship of organs in a human body; and
in step S23, using various organ phenotypic characteristics as nodes of the tree, and using the parent-child association relationships between various organ phenotypic characteristics as edges of the tree, thereby obtaining the topological relationship tree of the organ phenotypes.

In specific implementation, firstly, the medical image is acquired from the hospital PACS system through a DICOM protocol, and the medical image is queried and transmitted using a DICOM service class (such as C-FIND, C-MOVE), so as to ensure that the medical image is stored in the standard DICOM format; then, the text report is extracted from the PACS system or hospital information system through an HL7 protocol, and HL7 messages (such as ORU, ADT) are parsed so as to obtain text information such as diagnostic report; and finally, a collected DICOM image and HL7 text report are transmitted to local or quality control system, and data verification and preprocessing are performed so as to ensure data integrity and availability.

An organ phenotype extraction module 200, configured to construct a topological relationship tree of organ phenotypes according to the association of anatomical structures between various organ phenotypic characteristics in the medical image, and further determine the structural association graph of the organ phenotypes in the medical image based on the topological relationship tree and the distribution information of the organ contours in the organ phenotypic characteristics.

It should be noted that the organ phenotypic characteristics in the present disclosure refer to characteristics that can characterize an external morphology and structural state of organs in the medical image. The parent-child association relationships in the present disclosure refer to spatial subordinate relationships between organs in human anatomical structures, where a “parent” organ is an organ that is structurally superior or inclusive, and a “child” organ is an organ that is attached to or included in the “parent” organ. The parent-child association relationships can reflect hierarchical relationships between organs in spatial position and anatomical function. For example, the liver is a parent organ tissue of the gallbladder, and the lung is a parent organ of the fetus. Constructing the association between organs through such relationship helps to form a topological structure model with human anatomical logic. The topological relationship tree in the present disclosure refers to a directed tree data structure constructed based on the anatomical structures and spatial relationship between organs. The topological relationship tree is used to reflect spatial dependence between organs, anatomical subordination (anatomical hierarchies) and interconnected tissue characteristics.

In specific implementation, the pre-trained organ segmentation model (such as U-Net, nnUNet or DeepLab deep learning model) can be used to perform the organ segmentation on the medical images. By inputting the medical images in the DICOM format, the organ segmentation model outputs an organ label of each pixel and extracts phenotypic characteristics of various organs. Secondly, based on medical anatomical knowledge, the spatial position and anatomical proximity relationship between the organs are defined (such as a proximity relationship between the liver and gallbladder, a connection relationship between the heart and large blood vessels), the spatial position and anatomical proximity relationship are mapped into a relationship vector, and the relationship vector is used as the parent-child association relationship between various organ phenotypic characteristics. Finally, each organ phenotypic characteristic is used as the nodes of the tree, and the parent-child association relationships between the organ phenotypic characteristics are used as the edges of the tree, and the topological relationship tree of the organ phenotypes is constructed using graph theory or tree structure algorithm (such as minimum spanning tree or hierarchical clustering), which can intuitively represent anatomical structure association between organs. It should be noted that the embodiment of the present disclosure combines deep learning segmentation technology and anatomical knowledge to realize automatic construction of the organ topological relationship tree from the medical images.

In the present embodiment, determining the structural association graph of the organ phenotypes in the medical image based on the topological relationship tree and the distribution information of the organ contours in the organ phenotypic characteristics can be implemented through steps of:
extracting the distribution information of the organ contours in various organ phenotypic characteristics;
determining attribute information of various organ phenotypes according to the distribution information of various organ contours;
determining attribute relationships between various organ phenotypic characteristics through all the attribute information; and
adjusting the edges in the topological relationship tree through all the attribute relationships, and
using an adjusted topological relationship tree as the structural association graph of the organ phenotypes in the medical image.

It should be noted that the structural association graph in the present disclosure refers to an undirected relationship graph structure constructed based on organ phenotypic characteristic attributes, various nodes represent the phenotypic characteristics of different organs in a medical relationship image, and various edges represent connection between organs established based on the attribute relationships and structural relationships. The overall graph not only retains an anatomical hierarchy of the organs, but also reflects actual relation between the organs in morphology, space and function, for visualizing and analyzing an overall structural layout and interconnection of the organs, thus providing comprehensive and reliable data support for the medical imaging quality control or disease analysis.

In specific implementation, firstly, segmentation information of each organ is extracted using an existing medical image processing technology (such as deep learning-based segmentation network). A model used, such as U-Net, can accurately segment the organs of the medical image, thereby extracting an external boundary of each organ and obtaining the segmentation information of each organ (such as a modeled shape). Secondly, size, shape and spatial distribution parameters in the distribution information of various organ contours can be used as the attribute information of various organ phenotypes. Then, for each two organ phenotypes, an Euclidean distance of attribute information between the two organ phenotypes can be used as the attribute relationship between the two organ phenotypic characteristics, and then the attribute relationships between each two organ phenotypic characteristics can be obtained. Finally, a natural exponential function value of an opposite number of the attribute relationship between the two organ phenotypic characteristics (nodes) can be used as an adjustment coefficient of the edge between the two organ phenotypic characteristics (nodes), and then a product between the adjustment coefficient and a corresponding edge (i.e., the parent-child association relationship) is used as an adjusted edge in the topological relationship tree. All the adjusted edges can be obtained by the above method, then the edges before adjustment are replaced by all the adjusted edges, and then the topological relationship tree after the edge replacement is used as the structural association graph of the organ phenotypes in the medical image.

A medical entity association module 300, configured to determine the semantic structure graph of the medical entities in the text report through the logical association relationship between the medical entities and the contextual relationship of the clinical description statements in the text report.

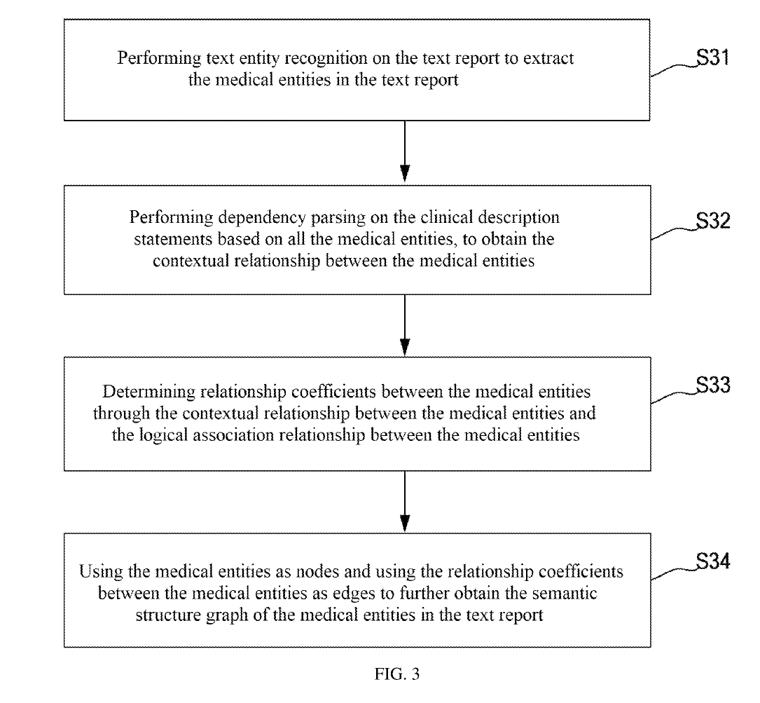

In the present embodiment, with reference to FIG. 3, the figure is an exemplary flowchart for determining the semantic structure graph in the embodiment of the present disclosure. In the present embodiment, determining the semantic structure graph of the medical entities in the text report through the logical association relationship between the medical entities and the contextual relationship of the clinical description statements in the text report can be implemented by steps as follows:
in step S31, performing text entity recognition on the text report to extract the medical entities in the text report;
in step S32, performing dependency parsing on the clinical description statements based on all the medical entities, to obtain the contextual relationship between the medical entities;
in step S33, determining relationship coefficients between the medical entities through the contextual relationship between the medical entities and the logical association relationship between the medical entities; and
in step S34, using the medical entities as nodes and using the relationship coefficients between the medical entities as edges to further obtain the semantic structure graph of the medical entities in the text report.

It should be noted that the medical entities in the present disclosure refer to words that have definite medical meanings in a medical text and can independently refer to specific medical concepts, typically including disease names, symptom manifestations, examination indicators, drug names, anatomical parts, surgical operations and pathological conditions; the relationship coefficient in the present disclosure is an indicator to measure strength of the relationship between the medical entities; and the semantic structure graph in the present disclosure refers to a graphical structure that uses the medical entities as nodes and connects various nodes through edges that represent semantic relationships between the medical entities, aiming to fully present semantic associations and logical relations between various medical entities in the text.

In specific implementation, firstly, an existing pre-trained entity recognition model (such as BioBERT or MedNER) can be used to perform entity recognition on the clinical description statements in the text report, and extract medical entity words related to diseases, symptoms, examination indicators and drugs therein. Secondly, the dependency parsing is performed on each clinical description statement in the text report using a dependency parsing-based tool (such as an SpaCy-based medical extension model or Stanza), a grammatical dependency relationship between various medical entities in the statement is clarified, and contextual relationships of subject-predicate-object, modification, and coordination are extracted. Then, combined with entity logical association in a medical knowledge graph (such as causal, concurrent, and subordinate relationships in UMLS or self-built medical knowledge base), the dependency relationship and logical association between each pair of medical entities are scored through a graph convolutional network (GCN) or a relational classifier (such as an RE model), and the score is used as the relationship coefficient between the medical entities, and the relationship coefficient can be used to measure the strength of the association between the medical entities. Finally, all extracted medical entities are used as nodes of the graph, and the relationship coefficients between the medical entities are used as edges of the graph, a graph construction tool (such as NetworkX) is used to construct the semantic structure graph of the medical entities, that is, all nodes and edges of all graphs can be substituted into the graph construction tool, and an output result is used as the semantic structure graph of the medical entities in the text report, thereby realizing visualization and structuralization of semantic associations.

A characteristic fusion module 400, configured to perform node-level alignment and fusion on the structural association graph of the organ phenotypes and the semantic structure graph of the medical entities to obtain an organ-entity characteristic association map.

In the present embodiment, performing the node-level alignment and fusion on the structural association graph of the organ phenotypes and the semantic structure graph of the medical entities to obtain the organ-entity characteristic association map can be implemented by steps of:
aligning corresponding nodes between the structural association graph of the organ phenotypes and the semantic structure graph of the medical entities to obtain multiple aligned and fused points; and
performing semantic matching on the aligned structural association graph and semantic structure graph to obtain characteristic association values of various aligned and fused points; and
determining the organ-entity characteristic association map through all the characteristic association values.

It should be noted that the aligned and fused points in the present disclosure refer to corresponding medical entity nodes that are semantically identical or similar in the structural association graph of the organ phenotypes and the semantic structure graph of the medical entities, and represented by nodes in the graphs; the characteristic association values in the present disclosure refer to quantitative indicators of semantic and structural association strength between organ phenotype nodes and medical entity nodes in the aligned and fused points; and the characteristic association map in the present disclosure is a graph structure configured to express and analyze multi-level and multi-dimensional association relationships between different medical entities and organ phenotypes, aiming to reveal semantic relation and structural relation between the entities through quantified characteristic association values.

In specific implementation, firstly, a graph matching algorithm (such as graph isomorphism detection based on adjacency matrix or similarity calculation method based on node embedding) can be used to align nodes that are semantically identical or similar in the structural association graph of organ phenotypes and the semantic structure graph of medical entities. GraphSAGE or Node2Vec is usually used to vectorize the nodes of the two graphs, and then node similarity is calculated by cosine similarity or Euclidean distance to screen out multiple aligned and fused points with high similarity. Secondly, a cross-graph neural network (such as Graph Matching Network, GMN) is used to perform characteristic comparison on a local structure and context around the aligned and fused points to achieve semantic fine-grained matching of the aligned and fused points, and the prior knowledge in the medical oncology (such as UMLS or self-built phenotype-entity mapping library) is further combined to calculate the characteristic association value of each aligned and fused point, and strength and type of relation between the organ phenotypes and the medical entities can be quantified through the characteristic association value. Finally, based on the characteristic association values of all aligned and fused points, a graph fusion strategy (such as weighted edge fusion or attention mechanism fusion) is used to integrate the two graphs (the structural association graph and the semantic structure graph) into a unified organ-entity characteristic association map. Through the organ-entity characteristic association map, systematic modeling of a multi-level and multi-dimensional semantic relationship between the organ phenotypes and the medical entities can be realized.

A quality control determination module 500, configured to perform quality control determination on the medical imaging data based on the characteristic association map, and output a quality control warning if it is determined that the medical imaging data has an imaging quality anomaly.

In the present embodiment, performing the quality control determination on the medical imaging data based on the characteristic association map can be implemented by steps of:
performing modal association evaluation on an imaging quality of the medical imaging data through the characteristic association map to obtain a quality evaluation value of the medical imaging data; and
determining that the medical imaging data has an imaging quality anomaly if the quality evaluation value is lower than a preset imaging anomaly threshold.

It should be noted that the quality evaluation value in the present disclosure is an indicator for measuring imaging data quality; and the preset imaging anomaly threshold in the present disclosure can be set to a value capable of effectively distinguishing between normal and abnormal imaging qualities by analyzing quality distribution of historical medical imaging data.

In specific implementation, firstly, a multimodal graph neural network (such as Graph Convolutional Network, GCN) can be used to analyze the characteristic association map, which contains the medical entities and organ phenotype information in the text report and the imaging data. The multimodal graph neural network can fuse data of different modalities through graph embedding technology, and combine the medical entities and characteristics extracted from the text report and the imaging data, so as to capture the association between the two. In a modal association evaluation stage, an interactive relationship between the imaging data and text data can be modeled using graph convolution operations, so as to generate a quality evaluation value, where the quality evaluation can reflect consistency between imaging quality and text description. In order to improve evaluation accuracy, an attention mechanism (such as Graph Attention Network, GAT) can also be introduced to weight information processing according to characteristic importance of each node, thus enhancing influence of key characteristics. Then, by comparing with the preset imaging anomaly threshold, if the quality evaluation value is lower than the preset imaging anomaly threshold, it is determined that the imaging data has an imaging quality anomaly. Through this determination method, the quality control system can automatically identify potential problems in the imaging, thereby improving the efficiency and accuracy of quality control.

It should be noted that in the present disclosure, outputting the quality control warning if it is determined that the medical imaging data has the imaging quality anomaly means that when the quality control determination module determines that the medical imaging data has the imaging quality anomaly, it will prompt relevant personnel through a warning notification that the imaging data has a quality problem and needs further inspection or processing. The warning is usually expressed in a visual or audible manner, such as a pop-up window prompt, color marking, alarm sound, etc., to ensure that the quality control personnel can find the problem in time and take corresponding measures.

It can be seen that the solutions of the present disclosure perform the node-level alignment and fusion on the structural association graph of the organ phenotypes and the semantic structure graph of the medical entities to obtain the organ-entity characteristic association map, and then perform the quality control determination on the medical imaging data based on the characteristic association map. Firstly, the organ phenotype extraction module systematically models inter-organ phenotypic characteristics by constructing the organ phenotype topological relationship tree, and combining the anatomical structure association, effectively solves the problem of incomplete understanding of organ structure in the prior art, and further generates the structural association graph through the organ contour distribution information, so as to realize accurate identification of hidden imaging anomalies such as contour loss or dislocation, and improve meticulousness of image layer quality control. Secondly, the medical entity association module constructs the semantic structure graph based on the contextual relationship and the logical association of the medical entities in the text report, to make up for a shortcoming of conventional methods that do not utilize text semantics for quality control verification, thus enabling imaging quality control results to have clinical context support, and enhancing clinical applicability and interpretability of quality control. The characteristic fusion module then fuses the structural association graph of the organ phenotypes with the semantic structure graph of the medical entities through the node-level alignment, establishes a cross-modal organ-entity characteristic association map, breaks through an information barrier between the imaging data and the text data, solves a problem of insufficient multimodal data fusion, and realizes information complementarity. Finally, the quality control determination module performs the quality control analysis based on the characteristic association map, thus being capable of integrating image quality and text semantic consistency, accurately identifying the imaging quality anomaly, avoiding deviation caused by single data source determination, and improving reliability and intelligence level of overall quality control determination.

In summary, the technical solutions of the present disclosure can realize multimodal association quality control of medical imaging data, thereby improving the reliability of data.

The present disclosure is described with reference to the flowcharts and/or block diagram of the method, device (system) and computer program product according to the embodiments of the present disclosure. It should be understood that each process and/or box in the flowcharts and/or block diagram, as well as a combination of processes and/or boxes in the flowcharts and/or block diagram, can be implemented by computer program instructions. These computer program instructions can be provided to a processor of a general-purpose computer, a special-purpose computer, an embedded processor, or other programmable data processing device to generate a machine, so that the instructions executed by the processor of the computer or other programmable data processing device generate a device for implementing functions specified in one or more processes in the flowcharts and/or one or more boxes in the block diagram.

Those ordinarily skilled in the art could understand that all or part of the steps in the various methods of the above embodiments can be completed by instructing relevant hardware through a program, and the program can be stored in a computer-readable storage medium, and the storage medium includes a read-only memory (ROM), a random access memory (RAM), a programmable read-only memory (PROM), an erasable programmable read-only memory (EPROM), a one-time programmable read-only memory (OTPROM), an electronically-erasable programmable read-only memory (EEPROM), a compact disc read-only memory (CD-ROM) or other optical disc storage, magnetic disk storage, magnetic tape storage, or any other computer-readable medium that can be used to carry or store data.

It should also be noted that the terms “include”, “comprise” or any other variations thereof are intended to cover non-exclusive inclusion, so that a process, method, commodity or device including a series of elements includes not only those elements, but also other elements not explicitly listed, or also includes elements inherent to such process, method, commodity or device. Without more restrictions, an element defined by the sentence “comprising a …” does not exclude the existence of other identical elements in the process, method, commodity or device including the element.

What is claimed is:

1. A system for medical imaging quality control and organ phenotype extraction, comprising:
a data acquisition module, configured to collect medical imaging data to be tested for quality control, wherein the medical imaging data comprises a medical image and a text report;
an organ phenotype extraction module, configured to construct a topological relationship tree of various organ phenotypes according to an association of anatomical structures between various organ phenotypic characteristics in the medical image, and then determine a structural association graph of the various organ phenotypes in the medical image based on the topological relationship tree and distribution information of various organ contours in the various organ phenotypic characteristics;
a medical entity association module, configured to determine a semantic structure graph of medical entities in the text report through a logical association relationship between the medical entities and a contextual relationship of clinical description statements in the text report;
a characteristic fusion module, configured to perform node-level alignment and fusion on the structural association graph of the various organ phenotypes and the semantic structure graph of the medical entities to obtain an organ-entity characteristic association map; and
a quality control determination module, configured to perform quality control determination on the medical imaging data based on the organ-entity characteristic association map, and output a quality control warning when the medical imaging data is determined to have an imaging quality anomaly.

2. The system according to claim 1, wherein the medical imaging data is collected from a hospital picture archiving and communication system (PACS) system.

3. The system according to claim 1, wherein a format of the medical image is a digital imaging and communications in medicine (DICOM) format.

4. The system according to claim 1, wherein a format of the text report is a health level seven (HL7) format.

5. The system according to claim 1, wherein constructing the topological relationship tree of the various organ phenotypes according to the association of the anatomical structures between the various organ phenotypic characteristics in the medical image comprises:
performing organ segmentation on the medical image using a pre-trained organ segmentation model, and extracting the various organ phenotypic characteristics;
defining parent-child association relationships between the various organ phenotypic characteristics based on a spatial position and anatomical proximity relationship of organs in a human body; and
using the various organ phenotypic characteristics as nodes of the topological relationship tree, and using the parent-child association relationships between the various organ phenotypic characteristics as edges of the topological relationship tree, to further obtain the topological relationship tree of the various organ phenotypes.

6. The system according to claim 1, wherein determining the structural association graph of the various organ phenotypes in the medical image based on the topological relationship tree and the distribution information of the various organ contours in the various organ phenotypic characteristics comprises:
extracting the distribution information of the various organ contours in the various organ phenotypic characteristics;
determining attribute information of the various organ phenotypes according to the distribution information of the various organ contours;
determining attribute relationships between the various organ phenotypic characteristics through all the attribute information; and
adjusting edges in the topological relationship tree through all the attribute relationships, and using an adjusted topological relationship tree as the structural association graph of the various organ phenotypes in the medical image.

7. The system according to claim 1, wherein determining the semantic structure graph of the medical entities in the text report through the logical association relationship between the medical entities and the contextual relationship of the clinical description statements in the text report comprises:
performing text entity recognition on the text report to extract the medical entities in the text report;
performing dependency parsing on the clinical description statements based on all the medical entities, to obtain the contextual relationship between the medical entities;
determining relationship coefficients between the medical entities through the contextual relationship between the medical entities and the logical association relationship between the medical entities; and
taking the medical entities as nodes and the relationship coefficients between the medical entities as edges, to further obtain the semantic structure graph of the medical entities in the text report.

8. The system according to claim 1, wherein performing the node-level alignment and fusion on the structural association graph of the various organ phenotypes and the semantic structure graph of the medical entities to obtain the organ-entity characteristic association map comprises:
aligning corresponding nodes between the structural association graph of the various organ phenotypes and the semantic structure graph of the medical entities to obtain a plurality of aligned and fused points;
performing semantic matching on an aligned structural association graph and semantic structure graph to obtain characteristic association values of the plurality of aligned and fused points; and
determining the organ-entity characteristic association map through all the characteristic association values.

9. The system according to claim 1, wherein performing the quality control determination on the medical imaging data based on the organ-entity characteristic association map comprises:
performing modality association evaluation on an imaging quality of the medical imaging data through the organ-entity characteristic association map to obtain a quality evaluation value of the medical imaging data; and
determining that the medical imaging data has the imaging quality anomaly when the quality evaluation value is lower than a preset imaging anomaly threshold.

10. The system according to claim 1, wherein outputting the quality control warning when the medical imaging data is determined to have the imaging quality anomaly means that when the quality control determination module determines that the medical imaging data has the imaging quality anomaly, a warning notification is configured to prompt a relevant personnel that the medical imaging data has a quality problem.

11. The system according to claim 5, wherein the various organ phenotypic characteristics are allowed for characterizing an external morphology and a structural state of organs in the medical image; the parent-child association relationships are spatial subordinate relationships between the organs in the anatomical structures; and the topological relationship tree is a directed tree data structure constructed based on the anatomical structures and a spatial relationship between the organs.

12. The system according to claim 1, wherein the structural association graph is an undirected relationship graph structure constructed based on organ phenotypic characteristic attributes.

13. The system according to claim 1, wherein the medical entities are words having definite medical meanings in a medical text and independently being medical concepts.

14. The system according to claim 8, wherein the plurality of aligned and fused points are medical entity nodes, wherein the medical entity nodes are semantically identical or similar in the structural association graph of the various organ phenotypes and the semantic structure graph of the medical entities, and represented by nodes in the structural association graph and the semantic structure graph; the characteristic association values are quantitative indicators of semantic and structural association strength between organ phenotype nodes and the medical entity nodes in the plurality of aligned and fused points; and the organ-entity characteristic association map is a graph structure configured to express and analyze multi-level and multi-dimensional association relationships between different medical entities and the various organ phenotypes.

15. The system according to claim 9, wherein the quality evaluation value is an indicator for measuring an imaging data quality; and the preset imaging anomaly threshold is set to a value allowed for effectively distinguishing between a normal imaging quality and an abnormal imaging quality by analyzing a quality distribution of historical medical imaging data.

16. The system according to claim 1, wherein the quality control warning is expressed in a visual manner or an audible manner.
